(19) United States
(12) Patent Application Publication
(10) Pub. No.: US 2025/0204859 A1
(43) Pub. Date: Jun. 26, 2025

(54) "SYSTEMS, METHODS, AND DEVICES FOR DETECTING PRESSURE ON A SURFACE"

(71) Applicant: Lenexa Medical Pty Ltd, Balwyn, Victoria (AU)

(72) Inventors: William Sing Ho YANG, Victoria (AU); Ajit RAVINDRAN, Victoria (AU); Blake Jordan FULLER, Victoria (AU); Yi YUAN, Victoria (AU); Arda YILMAZ, Victoria (AU)

(21) Appl. No.: 18/847,599

(22) PCT Filed: Mar. 17, 2023
(86) PCT No.: PCT/AU2023/050190

§ 371 Date: Sep. 16, 2024

(30) Foreign Application Priority Data
Mar. 18, 2022 (AU) 2022900676

(51) Int. Cl. A61B 5/00; G01L 1/18; G01L 9/00; G01L 9/06

(52) U.S. Cl. CPC A61B 5/6892; A61B 5/742; G01L 9/0055; G01L 9/06; A61B 2560/0443; A61B 2560/0462; A61B 2562/0247; A61B 2562/046; G01L 1/18

(57) ABSTRACT

Embodiments generally relate to a pressure sensing sheet system including a pressure sensing mat. The pressure sensing mat includes a piezoresistive material sandwiched between an at least one conductor array row and an at least one conductor array column, wherein each intersection of the conductor array row and the conductor array column defines a taxel; at least one sheet-edge row segment coupled to the at least one conductor array row; at least one sheet-edge column segment coupled to the at least one conductor array column; a sheet interface board connected to the at least one sheet-edge row segment and the at least one sheet-edge column segment, and configured to act as a fan-out; and a databox connected to the sheet interface board of the pressure sensing mat via signal wires and configured to measure each taxel of the pressure sensing mat.

### TECHNICAL FIELD

Embodiments generally relate to systems, methods, and devices for detecting pressure on a surface. In particular, embodiments relate to systems, methods, and devices of a pressure sensing sheet system used for detecting pressure applied to a surface.

### BACKGROUND

Pressure sensing sheets include conductor arrays comprising intersecting row and column conductors, and an addressing circuit which historically can be used to selectively address an intersection between each row conductor and each column conductor to measure changes in electrical characteristics at the intersection. The conductors may be in contact at the intersections, or may alternatively be electrically connected or separated by appropriate materials, including solids, liquids, and gases.

Conductor arrays are used in a wide and diverse range of applications, including medical applications, human machine interfaces and sensing applications, etc. For example, conductor arrays may be used in touch pads or touch screens where measured changes in inductance at an intersection caused by the proximity of a human finger can be used to locate and track the movement of the finger. Similarly, conductor arrays can be arranged on robot limbs to simulate touch sensing by measuring impedance which can be related to forces and pressure at each intersection.

In medical applications, addressing circuits can be used to measure changes in electrical characteristics at the intersections of a conductor array to determine changes in pressure. Pressure sensing sheets often comprise sensor nodes in the form of tactile sensor elements defined at the intersections between overlaid row conductors and column conductors arranged on the pressure sensing sheet. Each tactile sensor element is referred to as a taxel and the combination of sensor elements forms a taxel array. Each taxel within the taxel array can be monitored for changes in electrical characteristics using an addressing circuit to determine, for example, pressure applied to the pressure sensing sheet at the location of each taxel on the sheet.

Conventional pressure sensing sheets are typically commercially unviable due costs and current electrical designs resulting in poor implementation methods. It is desired to address or ameliorate one or more shortcomings or disadvantages associated with prior methods, systems, and designs of pressure sensing sheets, or to at least provide a useful alternative thereto.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

Some embodiments relate to a pressure sensing sheet system. The pressure sensing sheet system may include: a pressure sensing mat, wherein the pressure sensing mat may include: a piezoresistive material; an at least one conductor array row and an at least one conductor array column, wherein the piezoresistive material may be sandwiched between the at least one conductor array row and the at least one conductor array column, wherein each intersection of the conductor array row and the conductor array column defines a taxel; at least one sheet-edge row segment coupled to the at least one conductor array row; at least one sheet-edge column segment coupled to the at least one conductor array column; a sheet interface board connected to the at least one sheet-edge row segment and the at least one sheet-edge column segment, wherein the sheet interface board may be configured to act as a fan-out; and a databox connected to the sheet interface board of the pressure sensing mat via signal wires, where the databox may be configured to measure each taxel of the pressure sensing mat.

In some embodiments, the databox may be configured to measure each taxel by iterating across all conductor array rows and conductor array columns creating a circuit across each individual taxel.

In some embodiments, the taxel measurement value may be serialised, allowing for an at least 10 lead connection signal to the sheet interface board.

In some embodiments, the at least one sheet-edge row segment may drive a minimum of three and a maximum of eight conductor array rows.

In some embodiments, the at least one sheet-edge column segment may drive a minimum of three and a maximum of eight conductor array columns.

In some embodiments, both the at least one sheet-edge row segment and the at least one sheet-edge column segment may be designed to chain unidirectionally with additional sheet-edge row segments or sheet-edge column segments, respectively.

In some embodiments, the databox may be configured to communicate with an external server to transmit measurements for processing.

In some embodiments, the databox may dynamically determine the size of the conductor array.

In some embodiments, the databox may be configured to fault check the conductor array.

In some embodiments, the databox and the sheet interface board may be configured to measure multiple taxels at once to increase bandwidth.

In some embodiments, the at least one sheet-edge row segment and the at least one sheet-edge column segment may be configured to allow the databox to measure absolute resistance at each taxel of the conductor array.

In some embodiments, the at least one sheet-edge row segment and the at least one sheet-edge column segment may be configured to allow the databox to measure relative potential difference across each taxel of the conductor array.

In some embodiments, the databox may further be configured to display information representative of the pressure sensing sheet system.

In some embodiments, the databox may further be configured to output a display signal for the purpose of displaying information representative of to the pressure sensing sheet system.

Some embodiments relate to a method for determining pressure across a plurality of conductive rows and a plurality of conductive columns of a conductive fabric. The method may comprise: applying a first voltage supply to each row of the plurality of rows and each column of the plurality of columns of the conductive fabric; selecting a first of the plurality of rows and a first of the plurality of columns of the conductive fabric, wherein the selected row may be provided a second voltage supply greater than the first voltage supply; measuring an output of the selected column, the output may comprise an electrical signal; iterating the selected column from the first to a last of the plurality of columns, wherein for each iteration of the plurality of columns the measuring of the output of the selected column is repeated; iterating the selected row from the first to a last of the plurality of rows, wherein for each iteration of the plurality of rows the iteration of the columns is repeated; and determining pressures applied to the conductive fabric based on the measured outputs.

In some embodiments, the method may further comprise using the determined pressures applied to the conductive fabric to generate a heat map.

In some embodiments, the method may further comprise using the determined pressures applied to the conductive fabric to generate a plurality of heat maps.

Some embodiments relate to a method for determining a risk of pressure injuries in a patient using a plurality of conductive rows and a plurality of conductive columns of a conductive fabric. The method may comprise: applying a first voltage supply to each row of the plurality of rows and each column of the plurality of columns of the conductive fabric; selecting a first of the plurality of rows and a first of the plurality of columns of the conductive fabric, wherein the selected row may be provided a second voltage supply greater than the first voltage supply; measuring an output of the selected column, the output may comprise an electrical signal; iterating the selected column from the first to a last of the plurality of columns, wherein for each iteration of the plurality of columns the measuring of the output of the selected column is repeated; iterating the selected row from the first to a last of the plurality of rows, wherein for each iteration of the plurality of rows the iteration of the columns is repeated; determining pressures applied to the conductive fabric based on the measured outputs; generating, based on the pressures applied to the conductive fabric, a plurality of heat maps; and determining using a machine learning method, based on the plurality of heat maps, if the patient is at risk of developing a pressure injury.

Some embodiments relate to a method for determining a risk of a patient falling out of a bed using a plurality of conductive rows and a plurality of conductive columns of a conductive fabric. The method may comprise: applying a first voltage supply to each row of the plurality of rows and each column of the plurality of columns of the conductive fabric; selecting a first of the plurality of rows and a first of the plurality of columns of the conductive fabric, wherein the selected row may be provided a second voltage supply greater than the first voltage supply; measuring an output of the selected column, the output may comprise an electrical signal; iterating the selected column from the first to a last of the plurality of columns, wherein for each iteration of the plurality of columns the measuring of the output of the selected column is repeated; iterating the selected row from the first to a last of the plurality of rows, wherein for each iteration of the plurality of rows the iteration of the columns is repeated; determining pressures applied to the conductive fabric based on the measured outputs; generating, based on the pressures applied to the conductive fabric, a plurality of heat maps; and determining using a machine learning method, based on the plurality of heat maps, if the patient is at risk of falling out of the bed.

In some embodiments, the aforementioned methods may further include prior to applying the first voltage supply, a method for determining the number of conductive rows in the plurality of conductive rows. The method may comprise: applying an electrical pulse to a first row shift register of a plurality of row shift registers for a single clock cycle, wherein each bit of the plurality of row shift registers may be indicative of one of the plurality of conductive rows and the plurality of row shift registers may be connected in series; applying a plurality of row clock cycle signal to the plurality of row shift registers, wherein for every row clock cycle signal applied the electrical pulse shifts one bit in the plurality of row shift registers; receiving from a last row shift register of the plurality of row shift registers the electrical pulse; and determining the number of conductive rows in the plurality of rows based on the number of row clock cycle signals applied.

In some embodiments, the aforementioned methods may further include prior to applying the first voltage supply, a method for determining the number of conductive columns in the plurality of conductive columns. The method may comprise: applying an electrical pulse to a first column shift register of a plurality of column shift registers for a single clock cycle, wherein each bit of the plurality of column shift registers may be indicative of one of the plurality of conductive columns and the plurality of column shift registers may be connected in series; applying a plurality of column clock cycle signal to the plurality of column shift registers, wherein for every column clock cycle signal applied the electrical pulse shifts one bit in the plurality of column shift registers; receiving from a last column shift register of the plurality of column shift registers the electrical pulse; and determining the number of conductive columns in the plurality of columns based on the number of column clock cycle signals applied.

In some embodiments, the aforementioned methods may further include determining the number of conductive rows and the number of conductive columns based on data stored in a memory

In some embodiments, the aforementioned methods may further include a method for fault checking by determining if a fault is present based on the electrical pulse, wherein not receiving the electrical pulse after the determined number of clock cycle signals has been applied is indicative of a fault.

Some embodiments relate to a pressure sensing sheet system. The pressure sensing sheet system may include: a pressure sensing mat, wherein the pressure sensing mat may include: a piezoresistive material; an at least one conductor array row and an at least one conductor array column, wherein the piezoresistive material may be sandwiched between the at least one conductor array row and the at least one conductor array column creating a taxel where the at least one conductor array row and the at least one conductor array column intersect; a plurality of sheet-edge segments coupled to the at least one conductor array row and the at least one conductor array column; a sheet interface board connected to the plurality of sheet-edge segments, wherein the sheet interface board may be configured to act as a fan-out; and a databox connected to the sheet interface board of the pressure sensing mat via signal wires, wherein the databox may be configured to measure absolute resistance of each taxel of the pressure sensing mat.

Some embodiments relate to a pressure sensing sheet system. The pressure sensing sheet system may include: a conductor array, wherein the conductor array may include: a piezoresistive material; an at least three conductor array rows and an at least three conductor array columns; wherein the piezoresistive material may be sandwiched between the at least three conductor array rows and the at least three conductor array columns, creating a plurality of taxels where the at least three conductor array rows and the at least three conductor array columns intersect, wherein each intersection of a row and column defines a taxel; at least one sheet-edge row segment coupled to the at least three conductor array rows; at least one sheet-edge column segment coupled to the at least three conductor array columns; a sheet interface board, wherein the sheet interface board may be connected to the at least one sheet-edge row segment via a first wire connection and the at least one sheet-edge column segment via a second wire connection; and a databox connected to the sheet interface board, wherein the databox may be configured to measure each taxel of the conductor array.

### BRIEF DESCRIPTION OF DRAWINGS

FIG. 1 is a block diagram representation of a pressure sensing sheet system according to some embodiments;

FIG. 2 is a schematic representation of a portion of the conductor array of FIG. 1 according to some embodiments;

FIG. 3 is a schematic representation of the sheet-edge segment of FIG. 1 according to some embodiments;

FIG. 4 is a schematic representation of the sheet interface board of FIG. 1 according to some embodiments;

FIG. 5 is a block diagram of the databox of FIG. 1 according to some embodiments;

FIG. 6 is a schematic diagram of an example absolute pressure measurement configuration according to some embodiments;

FIG. 7 illustrates a simplified schematic of the transimpedence amplifier and taxel of FIG. 6 according to some embodiments;

FIG. 8 is a schematic diagram of an example relative pressure measurement configuration according to some embodiments;

FIG. 9 illustrates a schematic diagram of an example relative pressure measurement configuration of a portion of the pressure sensing sheet system of FIG. 1 according to some embodiments;

FIG. 10 illustrates a portion of sheet-edge segment of FIG. 3 when the pressure sensing sheet system is configured to measure relative pressure;

FIG. 11 illustrates an example matrix addressing timing diagram of the pressure sensing sheet system according to some embodiments;

FIG. 12 is a schematic diagram of an example absolute pressure measurement configuration according to some embodiments;

FIG. 13 is a simplified schematic diagram of the absolute pressure measurement configuration schematic diagram of FIG. 12;

FIG. 14A is an example heat map generate by the pressure sensing sheet system of FIG. 1 according to some embodiments;

FIG. 14B is an example heat map generated by the pressure sensing sheet system of FIG. 1 according to some embodiments;

FIG. 15 is a diagrammatic representation of a method for measuring vital signs of a patient according to some embodiments;

FIG. 16 is a diagrammatic representation of a method for measuring a height of a patient according to some embodiments;

FIG. 17 is a diagrammatic representation of a method for tracking movement of a patient according to some embodiments;

FIG. 18 is a diagrammatic representation of a method for determining a risk of a patient developing a pressure injury according to some embodiments;

FIG. 19 is a diagrammatic representation of a method for controlling an airbed; and

FIGS. 20A, 20B, and 20C show a diagrammatic representation of a method for controlling an airbed according to some embodiments.

### DESCRIPTION OF EMBODIMENTS

The conductor array of the embodiments may comprise technology and methods described in PCT Application No. PCT/AU2019/050976 filed 11 Sep. 2019 and titled “Addressing circuit for conductor arrays”, the contents of which are hereby incorporated by reference.

Described embodiments generally relate to systems, methods, and devices for detecting pressure on a surface. Particular embodiments relate to systems, methods, and devices of a pressure sensing smart sheet used for detecting pressure on a surface.

Throughout the specification the term ‘raster’ will be understood to mean iterating through rows and/or columns. This rastering method follows a row-lead scheme, where, for rows a to n and columns o to z, for each row a to n all columns o to z will be checked/scanned. For example, row a is selected and columns o to z are then scanned, then row b is selected and columns o to z are then scanned, for all rows a to n and columns o to z.

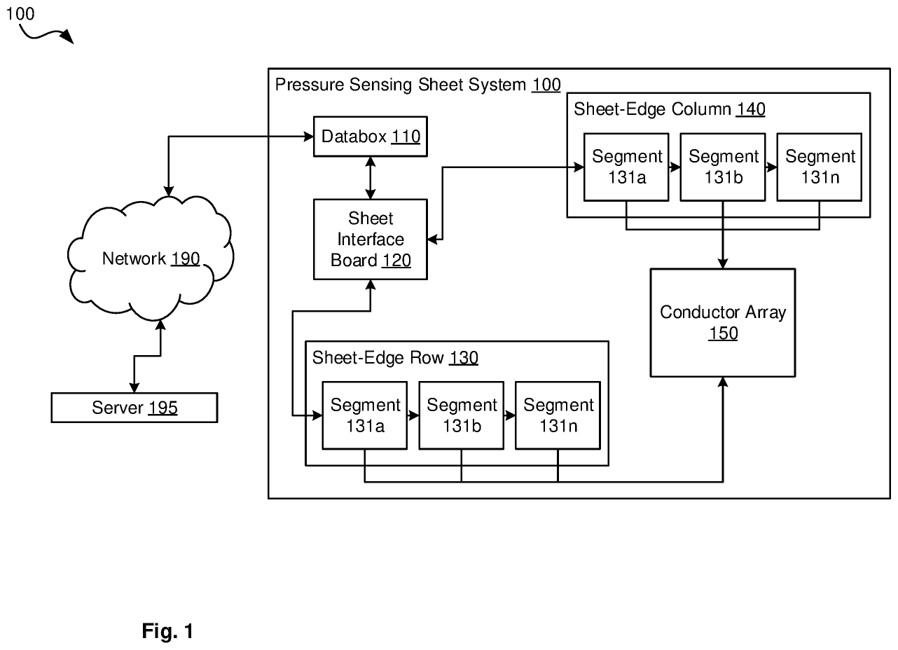

Referring to the drawings, FIG. 1 illustrates a block diagram of a pressure sensing sheet system 100 according to some embodiments. Pressure sensing sheet system 100 comprises a databox 110, sheet interface board 120, sheet-edge row 130 comprising sheet-edge segments 131a . . . n, sheet-edge column 140 comprising sheet-edge segments 131a . . . n, and conductor array 150. Sheet-edge segments 131a . . . n may be connected in series unidirectionally in their respective sheet-edge row 130 or sheet-edge column 140. In some embodiments, pressure sensing sheet system 100 may communicate with server 195 via network 190. Server 195 may be a physical server or a cloud-based server.

Network 190 may comprise one or more local area networks or wide area networks that facilitate communication between pressure sensing sheet system 100 and server 195. For example, according to some embodiments, network 190 may be the internet. However, network 190 may comprise at least a portion of any one or more networks having one or more nodes that transmit, receive, forward, generate, buffer, store, route, switch, process, or a combination thereof, etc. one or more messages, packets, signals, some combination thereof, or so forth. Network 190 may include, for example, one or more of: a wireless network, a wired network, an internet, an intranet, a public network, a packet-switched network, a circuit-switched network, an ad hoc network, an infrastructure network, a public-switched telephone network (PSTN), a cable network, a cellular network, a satellite network, a fibre-optic network, or some combination thereof.

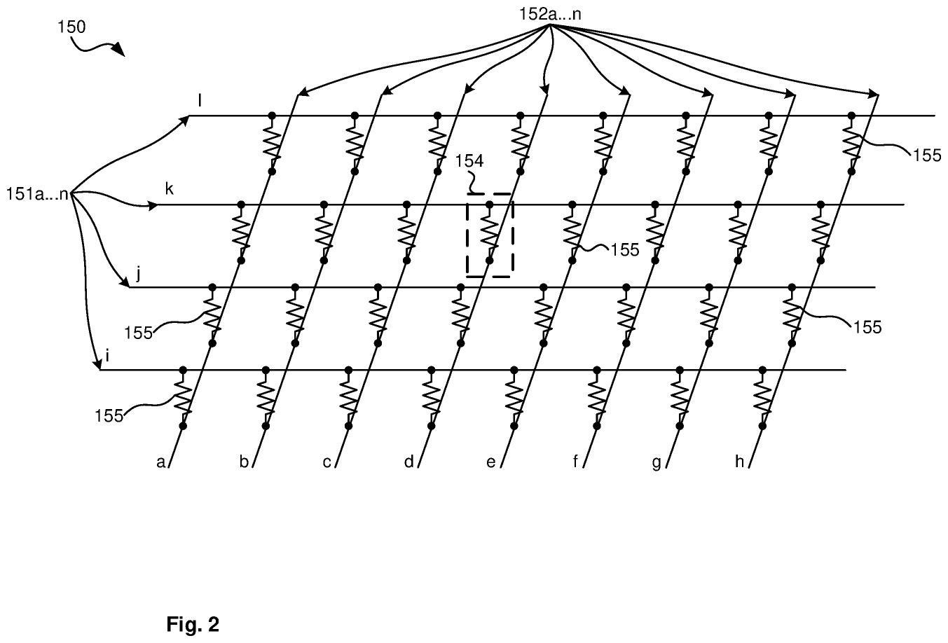

Illustrated in FIG. 2 is a portion of a conductor array 150 of the pressure sensing sheet system 100. The conductor array 150 comprises at least one conductor row 151, which may be referred to as row 151, at least one conductor column 152, which may be referred to as column 152, and a compression dependant resistive material 155. The rows 151a . . . n and columns 152a . . . m create a matrix, or taxel array. In some embodiments, the maximum number of rows 151a . . . n is 200 and the maximum number of columns 152a . . . m is 100. Each address, or taxel, in the matrix can be addressed by selecting the desired row 151n and column 151n. In some embodiments, the compression dependant material 155 may comprise a piezoresistive material, where the resistivity through the material may change depending on mechanical strain applied to the material. That is, when mechanical strain, or pressure, is increased at a taxel on the compression dependant material 155, the resistance measured through the material at that taxel increases. The compression dependant resistive material 155 is sandwiched between the rows 151a . . . n and the columns 152a . . . m, creating an electrical connection between the two layers of conductors as shown in FIG. 2. For example, an electrical current may travel along row k of conductor rows 151a . . . n, pass through the compression dependant material 155 at a conductor array taxel 154 and then travel along column d of conductor columns 152a . . . m.

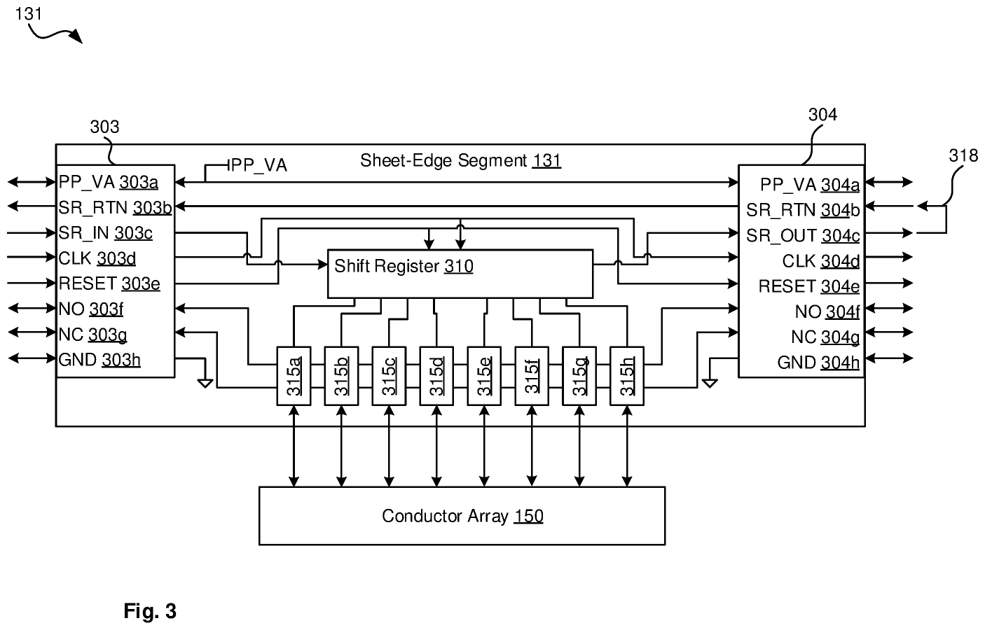

FIG. 3 shows a schematic diagram of a singular sheet-edge segment 131. Sheet-edge segment 131 comprises a shift register 310, at least three analogue switches 315a . . . h, segment inputs 303, and segment outputs 304. Segment inputs 303 and segment outputs 304 comprise pins 303a . . . h and pins 304a . . . h, respectively. In some embodiments, shift register 310 is a 74HC595 shift register. In some embodiments, analogue switches 315a . . . h are single-pole double-throw (SPDT) analogue switches. Each sheet-edge segment 131 may be capable of supporting at least three and at most eight conductor rows 151a . . . n or conductor columns 152a . . . m. Sheet-edge segments 131a . . . n may be connected in series unidirectionally, allowing an increase in the size of conductor array 150. For example, three sheet-edge segments 131a . . . n of sheet-edge row 130 are capable of supporting a total of 24 conductor rows 151a . . . n, and two sheet-edge segments 131a . . . n of sheet-edge column 140 are capable of supporting a total of 16 conductor columns 151a . . . n. Sheet-edge segments 131a . . . n are connected to either rows 151a . . . n or columns 152a . . . m via each analogue switch 315, where one analogue switch 315 connects to one row 151 or column 152 depending on whether sheet-edge segment 131 is in a sheet-edge row 130 or sheet-edge column 140.

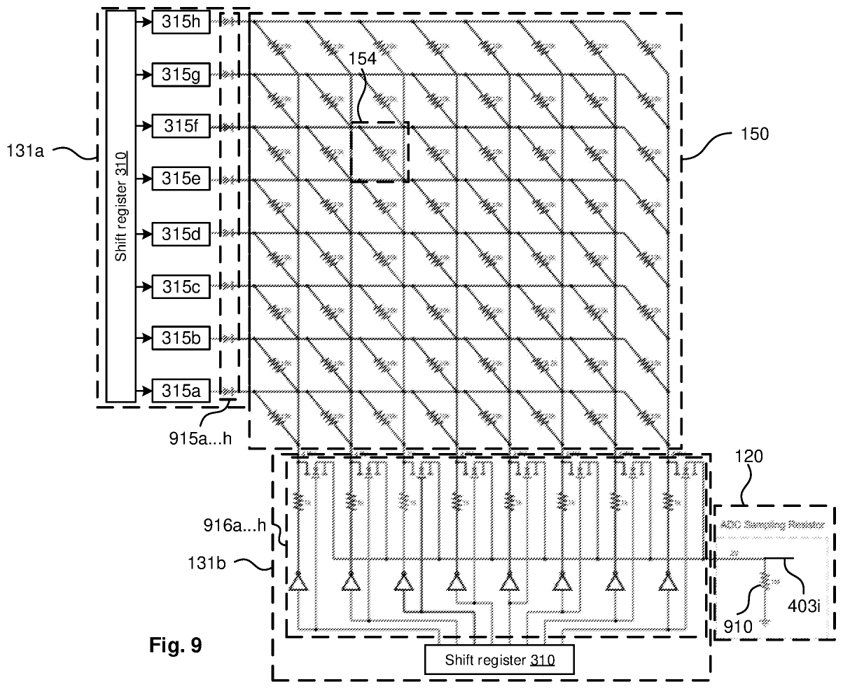

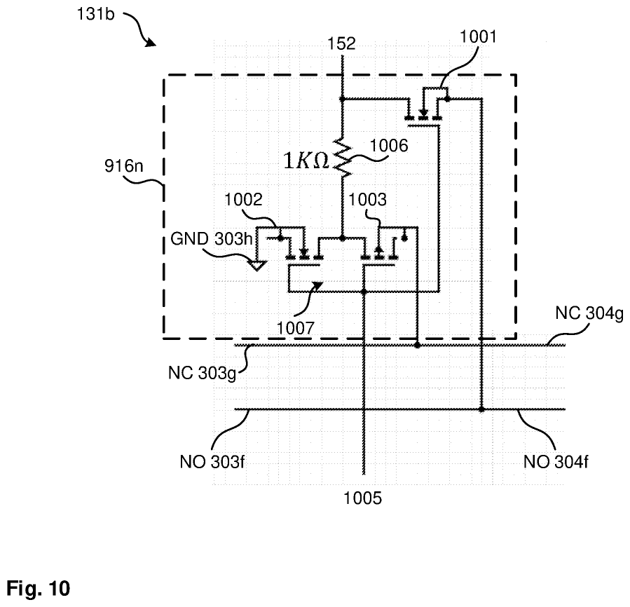

In alternate embodiments, the at least three analogue switches 315a . . . n of sheet-edge segments 131a . . . n of sheet-edge row 130 may include a diode at the connection point to their respective rows 151a . . . n, allowing current to flow in a single direction from sheet-edge segment 131 to conductor array 150, as described further on in relation to FIG. 9. The at least three analogue switches 315a . . . n of sheet-edge segments 131a . . . n of sheet-edge column 140 may instead be at least three n-type MOSFETs (NMOS) and complementary MOSFET (CMOS) inverters, as described further on in relation to FIG. 9 and FIG. 10.

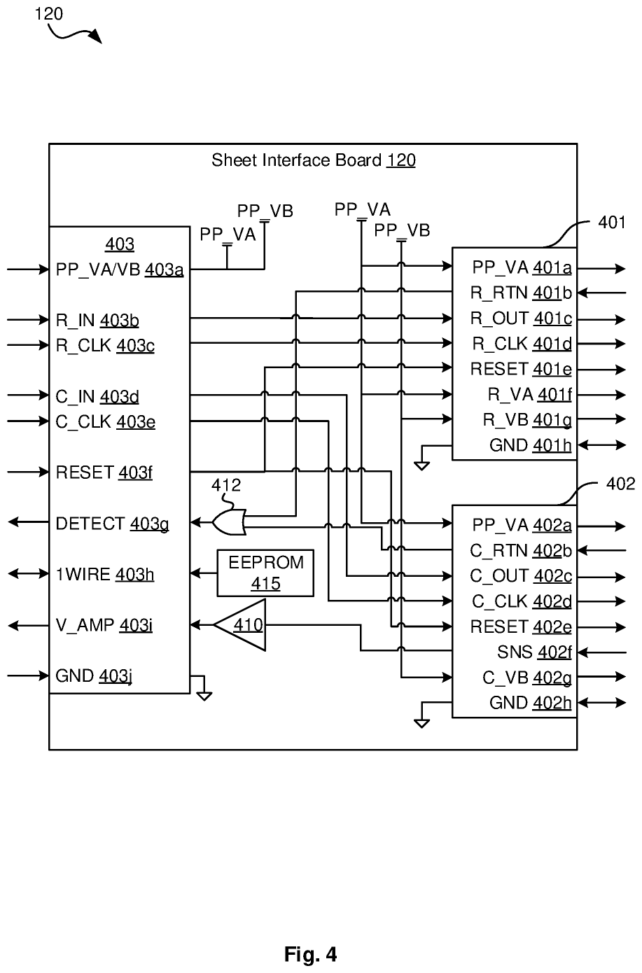

In some embodiments, pins 303a . . . h of segment inputs 303 may be connected to their corresponding pins 304a . . . h of segment outputs 304 of a preceding sheet-edge segment 131 in series within the same sheet-edge row 130 or sheet-edge column 140. In some embodiments, pins 304a . . . h of segment outputs 304 may be connected to their corresponding pins 303a . . . h of segment inputs 303 of a succeeding sheet-edge segment 131 in series within the same sheet-edge row 130 or sheet-edge column 140. That is, pin 303a may connect to pin 304a, and pin 304e may connect to pin 303e, for example. If a sheet-edge segment 131 is the first segment of sheet-edge segments 131a . . . n, segment inputs 303 are connected directly to sheet interface board 120 via row interface 401 for a sheet-edge segment 131a of a sheet-edge row 130, or column interface 402 for a sheet-edge segment 131a of a sheet-edge column 140 as described later in relation to FIG. 4.

In the illustrated embodiment, segment input 303 comprises eight pins 303a . . . h, namely, PP_VA 303a, SR_RTN 303b, SR_IN 303c, CLK 303d, RESET 303e, NO 303f, NC 303g, and GND 303h. PP_VA 303a is a power supply input providing power to the electrical components of sheet-edge segment 131. Throughout the present disclosure PP_VA may be referred to as the Vₐ rail or Vₐ reference voltage. SR_RTN 303b is a digital input/output comprising the final shift register loopback signal, used when detecting the number of sheet-edge segments 131a . . . n in a sheet-edge row 130 or sheet-edge column 140, as described further below. SR_IN 303c is a digital input comprising serial data shift register input, used to raster through rows 151a . . . n or columns 152a . . . m. CLK 303d is a digital shift register clock input that cycles the shift register on every pulse. RESET 303e is a digital input which acts as an asynchronous reset input. NO 303f is an analogue input/output (I/O) comprising a normally open pole of switch network signal. For sheet-edge segments 131a . . . n of sheet-edge row 130, NO 303f is connected to the Vₐ rail. For sheet-edge segments 131a . . . n of sheet-edge column 140, NO 303f is connected to a transimpedence amplifier (TIA) output. NC 303g is an analogue input comprising a normally closed pole of switch network, connected to PA_VB as described later in relation to FIG. 4. Throughout the present disclosure PP_VB may be referred to as the V_{b} rail or V_{b} reference voltage. GND 303h is a power signal providing a grounding connection.

In the illustrated embodiment, segment outputs 304 comprises eight pins 304a . . . h, namely, PP_VA 304a, SR_RTN 304b, SR_OUT 304c, CLK 304d, RESET 304e, NO 304f, NC 304g, and GND 304h. PP_VA 304a is an output of PP_VA 303a to the succeeding sheet-edge segment 131 in the series, if any. SR_RTN 304b is the input signal of the SR_RTN 303b output signal of any succeeding sheet-edge segment 131 in a sheet-edge row 130 or sheet-edge column 140. SR_OUT 304c is a digital output comprising shift register output from the last stage of the shift register 310. SR_OUT 304c lags SR_IN 303c by n cycles of CLK 303d, where n is the number of analogue switches 315a . . . n of sheet-edge segment 131. If the sheet-edge segment 131 is the last sheet-edge segment 131a . . . n of either sheet-edge row 130 or sheet-edge column 140 then the SR_OUT 304c output signal is connected to the SR_RTN 304b of the same sheet-edge segment 131, creating a loop as shown by arrow 318. Otherwise, the SR_OUT 304c output signal is connected to the SR_IN 303c input of the succeeding sheet-edge segment 131. CLK 304d is a digital output of the shift register index clock signal received by CLK 303d. RESET 304e is a digital output of the asynchronous reset signal received by RESET 303e. NO 304f is an output of the NO 303f signal, as previously described. NC 304g is the output of the NC 303g input, as previously described. GND 304h is a power signal providing a grounding connection. GND 304h may connect to the GND 303h input of the succeeding sheet-edge segment 131 in the series, if any.

Previously known solutions utilises a multiplexer/demultiplexer instead of sheet-edge segments 131 including shift registers 310. As such, previously known solutions require a plurality of control signals to determine a selected row and/or a selected column of a conductor array. This results in a large required number of connection leads from the conductor array to processing circuitry, and increases circuitry complexity. As the conductor array increases in size, more connection leads are required in previously known solutions. In embodiments of the present disclosure, the selected row and/or the selected column are iterated utilising a first clock signal for the rows and a second clock signal for the columns, requiring two connection leads for determining the selected row and the selected column. An increase in the size of conductor array 150 of the present disclosure does not require an increase in the required connection leads for iterating selected rows and columns.

FIG. 4 shows a schematic representation of the sheet interface board 120 of FIG. 1. Sheet interface board 120 comprises row interface (I/F) 401, column interface (I/F) 402, sheet interface (I/F) 403, a transimpedence amplifier (TIA) 410, a logic-OR gate 412, and an electrically erasable programmable read-only memory (EEPROM) 415. In some embodiments, the sheet interface board 120 is used to provide a signal fan-out between the sheet I/F 403 and the row I/F 401, and the sheet I/F 403 and the column I/F 402, as shown in FIG. 4. The TIA 410 may be used to convert a measured current through a taxel into a voltage, which is then transmitted to databox 110 via signal V_AMP 403i of sheet I/F 403. The logic-OR gate 412 may be used in part to determine the size of the conductor array 150, where, the logic-OR gate 412 may be used to allow two inputs to share the one output. The EEPROM 415 may be used to query the sheet interface board 120 to determine its design revision/variant and transmit this information to databox 110 via signal 1WIRE 403h of sheet I/F 403. In some embodiments, the EEPROM 415 may store data relating to the maximum possible size of a conductor array 150. In some embodiments sheet interface board 120 may include an analogue-to-digital converter (ADC) sampling resistor in place of the TIA 410.

Sheet I/F 403 may be connected to databox 110 via a 10 signal connector. Previously known solutions generally require an individual connection lead to the databox 110 for each of the total of the number of rows 151a . . . n and the number of columns 152a . . . n. That is, for each increase in either the number of rows 151a . . . n or the number of columns 152a . . . n, previously known solutions require an additional connection lead to the databox 110. In described embodiments of the present disclosure, an increase in either the number of rows 151a . . . n or the number of columns 152a . . . n does not result in an increase in the number of connection leads to the databox 110. Row I/F 401 may be connected to the first sheet-edge segment 131a of sheet-edge row 130 via 8 signals, where the letter, a to h, of each signal in row I/F 401 matches that of sheet-edge segment 131a segment inputs 303. For example, PP_VA 401a is connected to PP_VA 303a of the first sheet-edge segment 131a and R_VA 401f is connected to NO 303f of the first sheet-edge segment 131a of sheet-edge segments 131a . . . n in sheet-edge row 130. Column I/F 402 may similarly be connected to the first sheet-edge segment 131a of sheet-edge column 140 via 8 signals, where the letter, a to h, of each signal in column I/F 402 matches that of the sheet-edge segment 131a segment inputs 303. For example, SNS 402f is connected to NO 303f of the first sheet-edge segment 131a of sheet-edge segments 131a . . . n in sheet-edge column 140.

Row I/F 401 comprises signals PP_VA 401a, R_RTN 401b, R_OUT 401c, R_CLK 401d, RESET 401e, R_VA 401f, R_VB 401g, and GND 401h. Column I/F 402 comprises signals PP_VA 402a, C_RTN 402b, C_OUT 402c, C_CLK 402d, RESET 402e, SNS 402f, C_VB 402g, and GND 402h. PP_VA 401a, PP_VA 402a, and R_VA 401f are power outputs of the Vₐ rail received via power connection PP_VA/VB 403a of sheet I/F 403. Vₐ may act as a power supply input for sheet-edge segments 131a . . . n, and/or a reference voltage when sampling the conductor array 150. R_RTN 401b and C_RTN 402b are digital inputs of sheet-edge row 130 and sheet-edge column 140 respectively. In some embodiments, R_RTN 401b is used in part to determine the number of rows in conductor array 150, and C_RTN 402b is used in part to determine the number of columns in conductor array 150. R_OUT 401c and C_OUT 402c are digital outputs of the R_IN 403b and C_IN 403d signals received via sheet I/F 403. In some embodiments, R_OUT 401c is used in part to determine the number of rows in conductor array 150, and C_OUT 402c is used in part to determine the number of columns in conductor array 150. R_CLK 401d and C_CLK 402d are digital outputs of the R_CLK 403c and C_CLK 403e signals received via sheet I/F 403. RESET 401e and RESET 402e are digital outputs of the asynchronous RESET 403f signal received via sheet I/F 403, where they act to reset the row and column addresses of the sheet-edge row 130 and sheet-edge column 140 respectively. R_VB 401g and C_VB 4002g are power outputs of the of the V_{b} rail received via power connection PP_VA/VB 403a of sheet I/F 403. SNS 402f is an analogue signal comprising a current reading supplied by sheet-edge column 140. GND 401h and GND 402h is a power signal providing a grounding connection connected to databox 110 via GND 403j of sheet I/F 403.

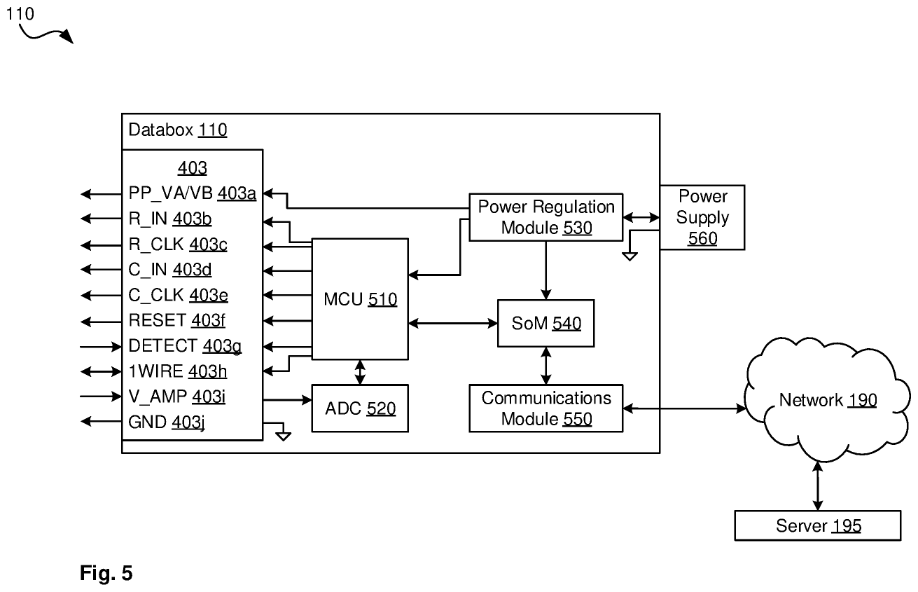

As illustrated in FIG. 5, databox 110 comprises an at least one microcontroller (MCU) 510, an at least one analogue-to-digital converter (ADC) 520, power regulation module 530, system on module (SoM) 540, communications module 550, power supply 560, and sheet interface I/F 403, as described in relation to FIG. 4. The MCU 210 manages collation of data from the ADC 220, as well as ensuring synchronisation between the ADC sampling and the matrix address. The ADC 520 acts to convert analogue signals received via V_AMP 403i of sheet I/F 403 to digital form. This conversion, or serialisation, allows for a single data connection per ADC 520 between the databox 110 and the sheet interface board 120. In some embodiments, databox 110 may comprise one or more ADCs 520 for converting a multitude of analogue outputs, where each additional ADC 520 increases the number of required connections between the databox 110 and the sheet interface board 120 by one. That is, databox 110 may comprise a plurality of ADC's 520 utilising interleaving to convert multiple taxel measurements simultaneously, for example.

Previously known solutions require the use of multiplexers and/or demultiplexers to communicate electrical signals to/from the databox 110 from/to the conductor array 150. As the size on the conductor array 150 of previously known solutions increases, circuit complexity also increases and larger or additional multiplexers/demultiplexers are required. Embodiments of the present disclosure do not require multiplexers/demultiplexers due to the sheet-edge segments 131a . . . n being configured to chain unidirectionally, reducing the number of connections to the databox 110 from the conductor array 150 and the overall complexity of the circuitry. Further, previously known solutions are limited to measuring a single taxel at a time, as use of a multiplexer/demultiplexer allows for either a single output from the conductor array 150 or input to the conductor array 150.

SoM 540 may be used to transmit collated data from the MCU 510, log data, and/or configuration details to server 195. In some embodiments, SoM 540 may be a SMARC 2.0, for example. In some embodiments, SoM 540 may be a COM Express Mini Type 10, for example. In some embodiments, SoM 540 may be a Qseven, for example. In some embodiments, SoM 540 may utilise any one of an x86, ARM, or AMD architecture. SoM 540 includes the necessary elements required to host an operating system. The operating system (OS) may be a windows OS, Linux OS, or Android OS, for example. In some embodiments, SoM 540 may include storage on board in the form of an “embedded Multi-Media Card” (eMMC). SoM 540 may require external storage connected via SATA, PCIe, or SD. In some embodiments, the SoM 540 is in communication with the MCU 510 via a high-speed serial interface. The absolute minimum baud rate in bits per second of the serial interface can be calculated using the following formula:

$$Baud Rate = n \times m \times {f}_{m} \times {w}_{A D C} \qquad ( 1 )$$

Where: m and n are the dimensions of the conductor array 150, fₘ is the desired scan rate of the conductor array 150 in frame samples per second, and w_{ADC} is the word length of an ADC sample in bits.

Power supply 560 is a direct current (DC) voltage power source. In some embodiments, power supply 560 may convert an alternating current (AC) power source to a DC power source. Power supply 560 connects to an external power source to supply DC voltage to the databox 110. Power supply 560 also provides a ground connection for all components within databox 110 and GND 403j of sheet I/F 403. Power regulation module 530 regulates the direct current (DC) voltage received via power supply 560, and supplies it to the components of databox 110. Power regulation module 530 also provides the Vₐ and V_{b} reference voltages, or voltage supplies, to PP_VA/VB 403a of sheet I/F 403.

Communications module 550 may allow for wired and/or wireless communication between databox 110 and external computing devices and components. Communications module 550 may facilitate communication via Bluetooth, USB, Wi-Fi, Ethernet, or via a telecommunications network, for example. According to some embodiments, communication module 550 may facilitate communication with external devices and systems via a network 190. In some embodiments, communications module 550 may implement either TCP or UDP-based data streaming. In some embodiments, communications module 550 may be embedded within SoM 540.

In some embodiments, sheet I/F 403 comprises a ten lead connection between the databox 110 and the sheet interface board 120. Sheet I/F 403 provides a direct connection between the components of databox 110 and the sheet-edge segments 131a . . . n, excluding DETECT 403g, 1WIRE 403h, and V_AMP 403i as illustrated in FIG. 4. For example, R_IN 403b directly connects from the MCU 510 to R_OUT 401c, which connects to SR_IN 303c of sheet-edge segment 131a of sheet-edge row 130. Whereas, DETECT 403g connects to sheet-edge segments 131a . . . n vis logic-OR gate 412, for example.

On initialisation of the pressure sensing sheet system 100, the size of the conductor array 150 is determined by the number of rows 151a . . . n and the number of columns 152a . . . m. The MCU 510 first determines the number of rows 151a . . . n by driving R_IN 403b high for a single pulse of R_CLK 403c. R_IN 403b is then driven low for every subsequent pulse of R_CLK 403c in the process of determining the number of rows 151a . . . n. The single high pulse of R_IN 403b is input to SR_IN 303c, and the signal of R_CLK 403c to CLK 303d, of the first sheet-edge segment 131a of sheet-edge row 130 via R_OUT 401c and R_CLK 401d of row I/F 401, respectively. MCU 510 will then raster through rows 151a . . . n by continuously pulsing R_CLK 403c on every clock cycle and counting the number of clock cycles that have passed, where, for every pulse of R_CLK 403c, shift register 310 of the current sheet-edge segment 131 is indexed by one. That is, the single high pulse received via SR_IN 303c moves along the shift register on every clock cycle of MCU 510. After a number of R_CLK 403c pulses equal to the number of analogue switches 315a . . . n on the current sheet-edge segment 131, the single high pulse is passed to the next sheet-edge segment 131 in the sheet-edge row 130 via SR_OUT 304c. This process is repeated for all sheet-edge segments 131a . . . n in sheet-edge row 130 until the final sheet-edge segment 131n is reached, where the single high pulse is input into SR_RTN 304b via connection 318, as shown in FIG. 3. The single high pulse is fed between sheet-edge segments 131n . . . a via the SR_RTN 303b and SR_RTN 304b connections of the sheet-edge segments 131a . . . n of sheet-edge row 130. Sheet-edge board 120 receives the single high pulse via R_RTN 401b, and outputs it to MCU 510 via the DETECT 403g connection after passing through the logic-OR gate 412. MCU 510 on receiving the single high pulse will determine the number of rows 151a . . . n by the number of clock cycles that have passed since initially outputting the single high pulse via R_IN 403b. For example, for fifty-three rows 151a . . . n, fifty-three MCU 510 clock cycles will have passed.

Following the determination of the number of rows 151a . . . n, the MCU 510 determines the number of columns 152a . . . m. To determine the number of columns 152a . . . m the same process as determining the number of rows 151a . . . n is followed. In the case of columns 152a . . . m however, MCU 510 drives C_IN 403d high for a single pulse of C_CLK 403e rather than R_IN 403b. C_IN 403d is then driven low for every subsequent pulse of C_CLK 403e in the process of determining the number of columns 152a . . . m. The single high pulse of C_IN 403d is input to SR_IN 303c, and the signal of C_CLK 403e to CLK 303d, of the first sheet-edge segment 131a of sheet-edge column 140 via C_OUT 402c and C_CLK 402d of column I/F 402, respectively. Sheet-edge board 120 receives the single high pulse via C_RTN 402b, and outputs it to the MCU 510 via the DETECT 403g connection after passing through the logic-OR gate 412. The MCU 510 on receiving the single high pulse will determine the number of columns 152a . . . m by the number of clock cycles that have passed since initially outputting the single high pulse via C_IN 403d. For example, for twenty-six columns 152a . . . m, twenty-six MCU 510 clock cycles will have passed. MCU 510 will have then determined the number of rows 151a . . . n and columns 152a . . . m of conductor array 150.

Pressure sensing sheet system 100 is able to raster through the rows 151a . . . n and columns 152a . . . m of the conductor array 150 by pulsing R_CLK 403c and C_CLK 403e. For every one pulse of R_CLK 403c emitted by the MCU 510, a number of pulses equivalent to the number of columns 152a . . . m, as determined on initialisation of the pressure sensing sheet system 100, will be emitted via C_CLK 403e. Following the raster of all columns 152a . . . m, R_CLK 403c will be pulsed again to address the next row 151 of rows 151a . . . n. This process will continue until all rows 151a . . . n and columns 152a . . . m for each row 151 have been rastered, indicating a single measurement cycle has passed.

Pressure sensing sheet system 100 may determine pressure (force) applied to a particular point of the conductor array 150, such as a conductor array taxel 154, by measuring either absolute resistance of a taxel or relative potential difference across a taxel. In some embodiments, pressure sensing sheet system 100 may be configured to measure absolute resistance of individual taxels. In some embodiments, pressure sensing sheet system 100 may be configured to measure relative potential difference across individual taxels. Due to the pressure sensing sheet system 100 measuring all taxels of the conductor array 150, complexity of the electronic system is significantly reduced. That is, the pressure sensing sheet system 100 does not need to only measure specific taxels, rather every taxel of conductor array 150 is measured in a single measurement cycle.

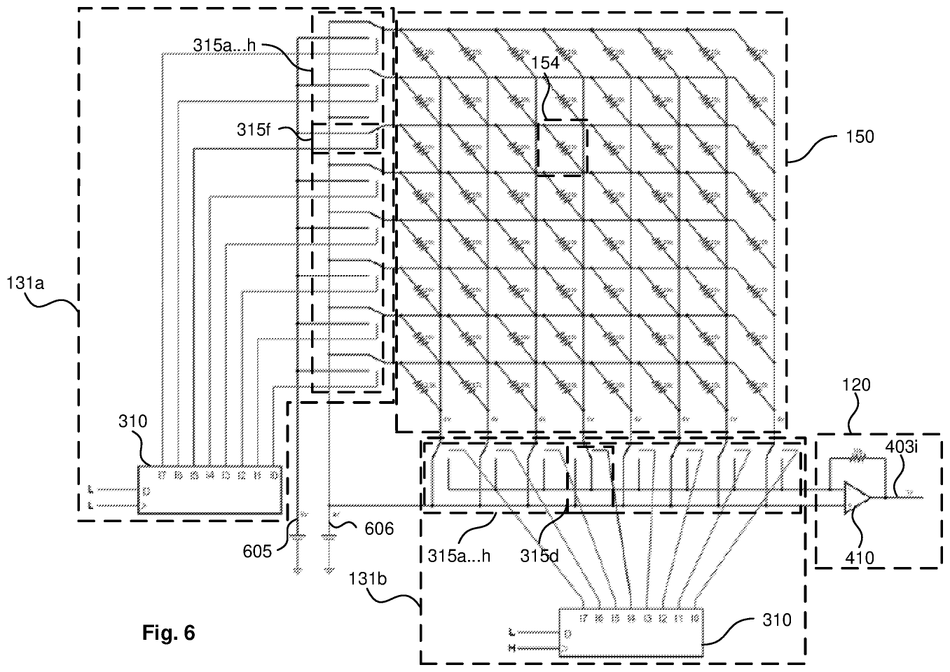

FIG. 6 illustrates a schematic diagram of an example absolute pressure measurement configuration of a portion of pressure sensing sheet system 100. FIG. 6 shows a conductor array 150, a portion of sheet-edge segment 131a and 131b of sheet-edge row 130 and sheet-edge column 140, respectively, and a portion of sheet interface board 120. Each sheet-edge segment 131a and 131b comprise a shift register 310 and analogue switches 315a . . . h. Power inputs, or voltage supplies, Vₐ and V_{b} are denoted by 605 and 606 respectively. The portion of sheet interface board 120 shown comprises, the TIA 410 and V_AMP 403i output to the MCU 510. In the example of FIG. 6, row 151f, or row 6, and column 151d, or column 4, are being addressed. That is, the absolute resistance of the conductor array taxel 154 of row 6 and column 4 is being measured.

All rows 151a . . . n and columns 152a . . . m of conductor array 150 are normally connected to V_{b} when not being addressed. When addressing row 151n, the shift register 310 of the corresponding sheet-edge segment 131 is outputting a high signal to the analogue switch 315n that is connected to the row 151n. Similarly, when addressing column 152n, the shift register 310 of the corresponding sheet-edge segment 131 is outputting a high signal to the analogue switch 315n that is connected to the column 152n. The high signal input to the analogue switch 315n for both the row 151n and column 152n changes the state of their respective analogue switches 315n from normally closed (NC) to normally open (NO). That is, the analogue switches 315a . . . n of sheet-edge segment 131 toggle from inputs NC 303g to NO 303f when powered, and from NO 303f to NC 303g when not powered. Referring to FIG. 6, when analogue switch 315f of sheet-edge segment 131a is toggled, row 6 of conductor array 150 switches from a V_{b} power source, or voltage supply, to a Vₐ power source, or voltage supply. When analogue switch 315d of sheet-edge segment 131b is toggled, column 4 of conductor array 150 switches from a V_{b} power source to a V_{TIA} output 403i. All other rows 151a . . . n and columns 151a . . . n are still connected to the V_{b} power source. The voltage output V_{TIA} can be read and then the absolute resistance of the addressed conductor array taxel 154 can be calculated by MCU 510.

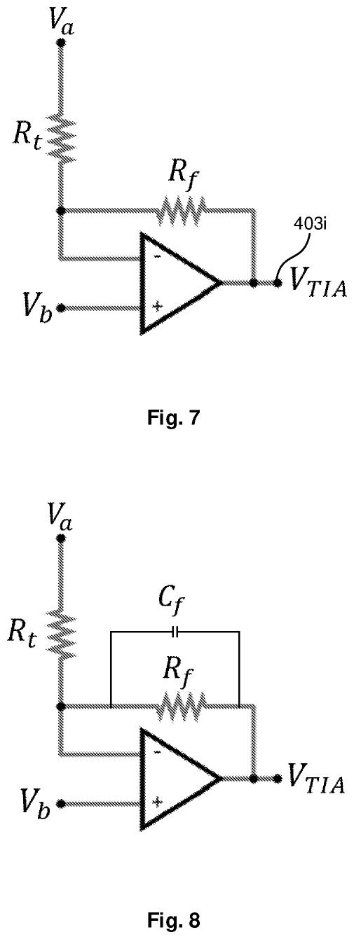

FIG. 7 shows a simplified schematic of the absolute resistance measurement schematic diagram of FIG. 6, focusing on the taxel of row 6 and column 4. The taxel resistance Rₜ as a function of the output voltage (V_{TIA}) at 403i can be derived in the following way:

$$\frac{{V}_{a} - {V}_{b}}{{R}_{t}} = \frac{{V}_{b} - {V}_{TIA}}{{R}_{f}} \qquad ( 2 )$$

$$\frac{{R}_{f}}{{R}_{t}} = \frac{{V}_{a} - {V}_{b}}{{V}_{b} - {V}_{TIA}} \qquad ( 3 )$$

$${R}_{t} = \frac{( {V}_{a} - {V}_{b} ) {R}_{f}}{{V}_{b} - {V}_{TIA}} \qquad ( 4 )$$

The above equation 3 models the DC response of the TIA 410. The taxel resistance Rₜ can be calculated as Vₐ, V_{b}, and R_{f} are known values, and V_{TIA} is measured. In implementation, an additional feedback capacitor (C_{f}) will be required in parallel with the feedback resistor (R_{f}) to stabilise the output V_{TIA}, as shown in FIG. 8. The resulting equation for calculating the resistance of a particular taxel Rₜ is:

$${R}_{t} = \frac{( {V}_{a} - {V}_{b} ) Z}{{V}_{b} - {V}_{TIA}} \qquad ( 5 )$$

$$Where : Z = \frac{1}{\frac{1}{{R}_{f}} + j ω {C}_{f}}$$

$$ω = 2 π f$$

$$and f is the frequency in Hertz ( Hz ) of the input signal .$$

The value of the feedback capacitor (C_{f}) in equation 4 is dependent on the bandwidth of the measured signal, the parasitic capacitance of the operational amplifier within the TIA 410, the capacitance of the piezoresistive material 155, and the capacitance of the supporting circuitry of the pressure sensing sheet system 100. The calculation of taxel resistance Rₜ is performed for every conductor array taxel 154 in conductor array 150 in a single full measurement cycle by rastering all rows 151a . . . n and columns 152a . . . m.

FIG. 9 illustrates a schematic diagram of an example relative pressure measurement configuration of a portion of pressure sensing sheet system 100. FIG. 8 shows a conductor array 150, a portion of sheet-edge segment 131a and 131b of sheet-edge row 130 and sheet-edge column 140, respectively, and a portion of sheet interface board 120. Sheet-edge segment 131a comprises, a multitude of analogue switches 315a . . . n in series with diodes 915a . . . h. Sheet-segment 131b comprises, a multitude of N-channel MOSFET (NMOS) and complementary MOSFET (CMOS) blocks 916a . . . h. The portion of sheet interface board 120 shown comprises, an ADC sampling resistor 910 and V_AMP 403i output to the MCU 510. In the example of FIG. 9, row 151f, or row 6, and column 151c, or column 3, are being addressed. That is, the relative potential, or voltage drop, across conductor array taxel 154 of row 6 and column 3 is being measured.

FIG. 10 illustrates a portion of sheet-edge segment 131b of sheet-edge column 140 when pressure sensing sheet system 100 is in a relative pressure measurement configuration. Input 1005 is a shift register bit input from shift register 310. 152 denotes a connection to a column 152 of columns 152a . . . m. NMOS 1001 and NMOS 1002 are N-channel MOSFETs. PMOS 1003 is a P-channel MOSFET, where the NMOS 1002 and the PMOS 1003 are combined to create a complementary MOSFET (CMOS). The source of the NMOS 1002 is a connection to the ground 303h of sheet-edge segment 131b, and the source of PMOS 1003 is connected to input NC 304g, the V_{b} rail, of sheet-edge segment 131b. The gates of the NMOS 1001, NMOS 1002, and PMOS 1003 are connected to the shift register bit input 1005. The drains of the NMOS 1002 and PMOS 1003 are connected to the column 152 and the drain of the NMOS 1001 via a resistor 1006. CMOS 1007 acts as an inverter to invert the signal received via the source of PMOS 1003 when the gates of NMOS 1002 and PMOS 1003 are driven high. That is, when shift register bit input 1005 is driven low the CMOS 1007 will output the signal supplied by NC 303g. When shift register bit input 1005 is driven high the CMOS 1007 will create a sink path through the inverter. The source of the NMOS 1001 is connected to output NO 303f of sheet-edge segment 131b, the drain of the NMOS 1001 is connected to its respective column 152 and the inverted signal output from the CMOS 1007.

Referring back to FIG. 9, all rows 151a . . . n and columns 152a . . . m of conductor array 150 are normally connected to V_{b} when not being addressed. That is, all unselected rows 151a . . . n and columns 152a . . . m float to the same potential, eliminating leakage paths. When addressing row 151n, the shift register 310 of the corresponding sheet-edge segment 131 is outputting a high signal to a to the diode 915n that is connected to the row 151n. Similarly, when addressing column 152n, the shift register 310 of the corresponding sheet-edge segment 131 is outputting a high signal to the NMOS and CMOS inverter block 916n that is connected to the column 152n. The high signal input to the analogue switch 315n for the row 151n changes the state of the respective analogue switch 315n from normally closed (NC) to normally open (NO). That is, the analogue switches 315a . . . n of sheet-edge segment 131 of sheet-edge row 130 toggle from inputs NC 303g to NO 303f when powered, and from NO 303f to NC 303g when not powered. As shown in FIG. 9, when analogue switch 315f of sheet-edge segment 131a is toggled, row 6 of conductor array 150 switches from a V_{b} power source to a Vₐ power source. The high signal input to the NMOS and CMOS inverter block 916n for column 152n activates the gate of the NMOS 1001 allowing current to pass through, and activates the gates of the CMOS 1007 to create a current sink path. That is, when the NMOS and CMOS inverter block 916a . . . n of sheet-edge segment 131 of sheet-edge column 140 is powered, NMOS 1001 of 916n switches from blocking current flow to allowing current to flow from the drain to the source, and CMOS 1007 of 916n switches from outputting the input received via NC 303g to instead creating a current sink path connected to ground 303h. All other rows 151a . . . n and columns 151a . . . n are still connected to the V_{b} power source. The relative potential difference across the conductor array taxel 154 of row 6 and column 3 can then be sampled by the ADC 520 via output 303f.

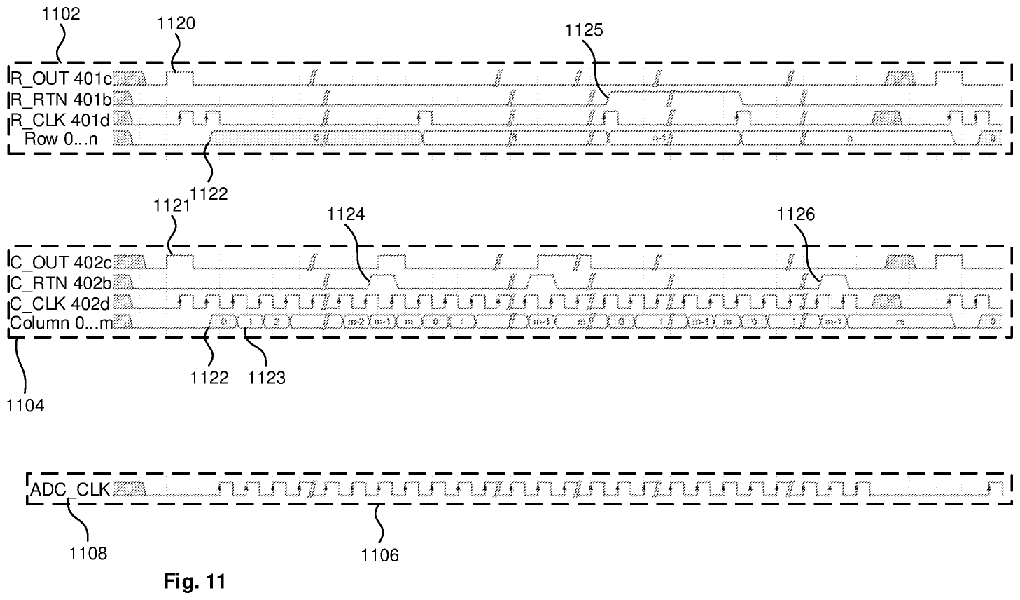

FIG. 11 illustrates an example matrix addressing timing diagram. The diagram includes a sheet-edge row timing portion 1102, a sheet-edge column timing portion 1104, and an ADC timing portion 1106. Sheet-edge row timing portion 1102 comprises signal timing diagrams for R_OUT 401c, R_RTN 401b, and R_CLK 401d, and the active Row 0 . . . n. Sheet-edge column timing portion 1104 comprises signal timing diagrams for C_OUT 402c, C_RTN 402b, and C_CLK 402d, and the active Column 0 . . . m. ADC timing portion 1106 includes a signal timing diagram for ADC_CLK 1108.

To begin a full measurement cycle, or complete raster, of the conductor array 150 the MCU 510 will drive R_OUT 401c high for a single clock cycle of R_CLK 401d at 1120. At the same time at 1121, MCU 510 will also drive C_OUT 402c high for a single clock cycle of C_CLK 402d. On the falling edge of the first clock cycle of R_CLK 401d and C_CLK 402d, MCU 510 will drive ROUT 401c and C_OUT 402c low. R_OUT 401c will be driven low for the remainder of the full raster of conductor array 150. C_OUT 402c will be driven low until all columns 152a . . . m for a single row 151 have been rastered. The R_OUT 401c and C_OUT 402c signals, or bits, are input into the shift register 310 of the first sheet-edge segment 131 in their respective sheet-edge row 130 or sheet-edge column 140. At 1122, on the rising edge of the next clock cycle of R_CLK 401d and C_CLK 402d the input bit of the sheet-edge row 130 and the sheet-edge column 140 is cycled to select the first register of their respective shift register 310. That is, active Row 0 and active Column 0 are being addressed. MCU 510 will stop pulsing R_CLK 401d with clock cycles, while continuing to pulse signals to C_CLK 402d. On the rising edge of every C_CLK 402d clock cycle the input bit of shift register 310 of sheet-edge column 140 cycles to the next register. That is, for every rising edge of the clock cycle of C_CLK 402d the shift register 310 will address the next active Column 0 . . . m. For example at 1123, after the third clock cycle of C_CLK 402d, the input bit of the shift register 310 of sheet-edge column 140 is selecting active Column 1.

The clock of C_CLK 402d will continue to cycle and the input bit of sheet-edge column 140 will shift along shift register 310 of each sheet-edge segment 131 in sheet-edge column 140 on each rising edge of each clock cycle. That is, when the input bit reaches register seven of a shift register 310, the shift register 310 will output a high signal, or a new input bit, to the respective ROUT 401c and C_OUT 402c outputs. This new input bit is the input to either the shift register 310 of the next sheet-edge segment 131, or if the shift register 310 is of the last sheet-edge segment 131 the signal is input to the MCU 510 to signal the end of a rows 151a . . . n or columns 152a . . . m cycle. When a succeeding shift register 310 is present, the input bit is input into the shift register 310 on the rising edge of the clock cycle that the eighth register of the preceding shift register 310 is selected. On the rising edge of the next clock cycle the preceding shift register 310 is no longer addressing a row 151 or column 152 and the input bit of the succeeding shift register 310 is cycled to select the first register and to address the next row 151 or column 152. This allows for a seamless transition when moving from addressing the last row 151 or column 152 of a preceding sheet-edge segment 131 to addressing the first row 151 or column 152 of a succeeding sheet-edge segment 131.

At 1124, on the transition between addressing active Column m-2 and active Column m-1 the C_RTN 402b output is driven high and the signal input to the MCU 510. The MCU 510 will then pulse the clock signal of R_CLK 401d on the second clock cycle after receiving the signal. That is, when all columns 151a . . . n in conductor array 150 have been addressed for a row 151, the MCU 510 will cycle to the next row 151 in conductor array 150. This process allows the MCU 510 to raster through all columns 152a . . . m of all rows 151a . . . n. At 1125, when the second last active Row n-1 is addressed the MCU 510 receives a signal via R_RTN 401b indicating that there is one row 151, active Row n, left to be sampled in the conductor array 150. The MCU 510, after cycling to the final active Row n via clock signal R_CLK 401d, will know that on receiving a signal via C_RTN 402b at 1126, that the entire conductor array 150 will have been sampled after the next clock cycle of C_CLK 402d.

The ADC_CLK 1108 clock cycle is inverted to that of the C_CLK 402d clock cycle. That is, for every falling edge of the clock cycle of C_CLK 402d the ADC_CLK 1108 has a rising edge of its clock cycle, and vice versa. For every rising edge of the ADC_CLK 1108 clock cycle, the ADC 520 samples the output VAMP 403i signal received from the sheet-edge column 140 and outputs it to the MCU 510 for processing. This rastering and sampling method is the same for both the absolute pressure measurement and the relative pressure measurement. In some embodiments, the rastering method is continuously repeated to detect changes in pressure applied to the pressure sensing sheet system 100.

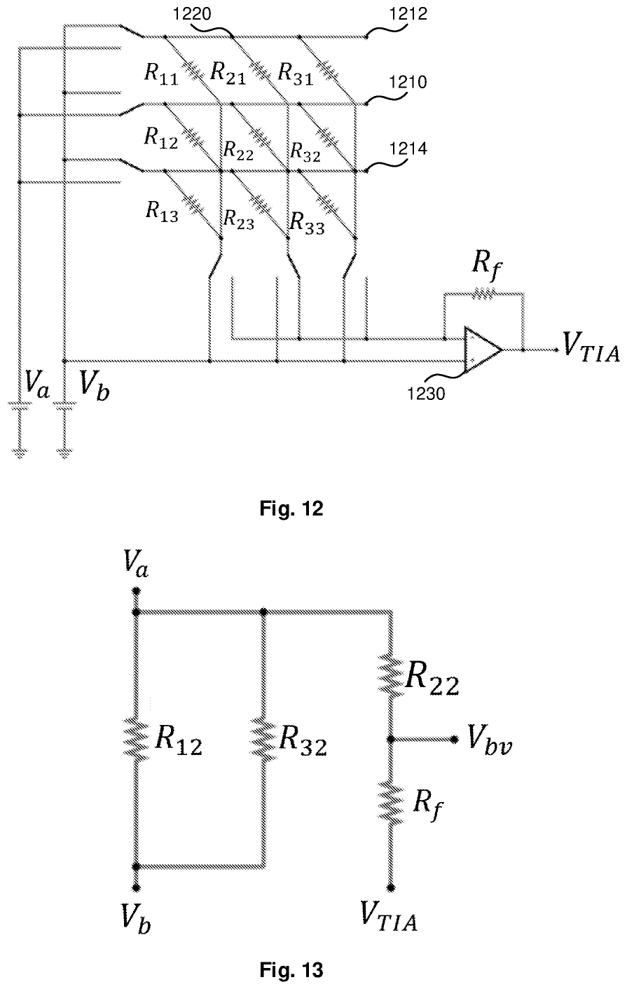

FIG. 12 illustrates a schematic diagram of an example absolute pressure measurement configuration. In FIG. 12, active row 1210 and active column 1220 are active for sampling. This allows a simplification of the circuit by eliminating all elements with zero potential difference. That is, all elements in row 1212 and row 1214 can be removed resulting in the simplified schematic diagram shown in FIG. 13.

As shown in FIG. 13, there is a parasitic consumption between Vₐ and V_{b} associated with the active row 1210. In the example of FIG. 13, the elements contributing to this consumption are R₁₂ and R₃₂. In some embodiments, the elements contributing to this consumption will be all elements in the active row 1210 that are not being sampled by the active column 1220. The general equation to calculate the parasitic consumption P_{RP} is:

$${P}_{RP} = \frac{( m - 1 ) {( {V}_{a} - {V}_{b} )}^{2}}{R} \qquad ( 6 )$$

$$Where : m is the width of the row , and$$

$$R is the minimum expectable resistance of the element .$$

In addition to parasitic consumption associated with the active row 1210, there is also a power loss associated with the element being sampled, in this case R₂₂. Due to the nature of the virtual voltage, V_{bv}, created by the transimpedence amplifier 1230, the consumption of the sampled element R₂₂ can be combined with P_{RP} to determine the power consumption of the conductor array 150, P_{M}:

$${P}_{M} = {P}_{RP} + \frac{{( {V}_{a} - {V}_{b} )}^{2}}{R} \qquad ( 7 )$$

$${P}_{M} = \frac{{m ( {V}_{a} - {V}_{b} )}^{2}}{R} \qquad ( 8 )$$

The majority of power delivered to the conductor array 150 via Vₐ is returned to the supply via V_{b}. However, Vₐ is still required to be capable of supplying the full power or current to conductor array 150. The total power burden on Vₐ can be expressed as:

$${P}_{A} = {P}_{M} + {P}_{B} + {P}_{TIA} \qquad ( 9 )$$

$$Where : {P}_{B} is the power returned to {V}_{b} , and$$

$${P}_{TIA} is the power sunk by the transimpedence amplifier 1230.$$

The power sunk by the transimpedence amplifier 1230 can be modelled as:

$${P}_{TIA} = \frac{( {V}_{bv} - {V}_{TIA} ) {V}_{bv}}{{R}_{f}} \qquad ( 10 )$$

$${P}_{TIA} = \frac{( {V}_{b} - {V}_{TIA} ) {V}_{b}}{{R}_{f}} \qquad ( 11 )$$

The power returned to V_{b} can be modelled as:

$${P}_{B} = \frac{{V}_{b} {P}_{R P}}{{V}_{a} - {V}_{b}} \qquad ( 12 )$$

$${P}_{B} = \frac{( m - 1 ) ( {V}_{a} - {V}_{b} ) {V}_{b}}{R} \qquad ( 13 )$$

Combining equations 7, 10, and 12 results in the total power burden on Vₐ as shown in equation 8, resulting in:

$${P}_{A} = \frac{{m ( {V}_{a} - {V}_{b} )}^{2}}{R} + \frac{( m - 1 ) ( {V}_{a} - {V}_{b} ) {V}_{b}}{R} + \frac{( {V}_{b} - {V}_{TIA} ) {V}_{b}}{{R}_{f}} \qquad ( 14 )$$

$${P}_{A} = \frac{( m {V}_{a} - {V}_{b} ) ( {V}_{a} - {V}_{b} )}{R} + \frac{( {V}_{b} - {V}_{TIA} ) {V}_{b}}{{R}_{f}} \qquad ( 15 )$$

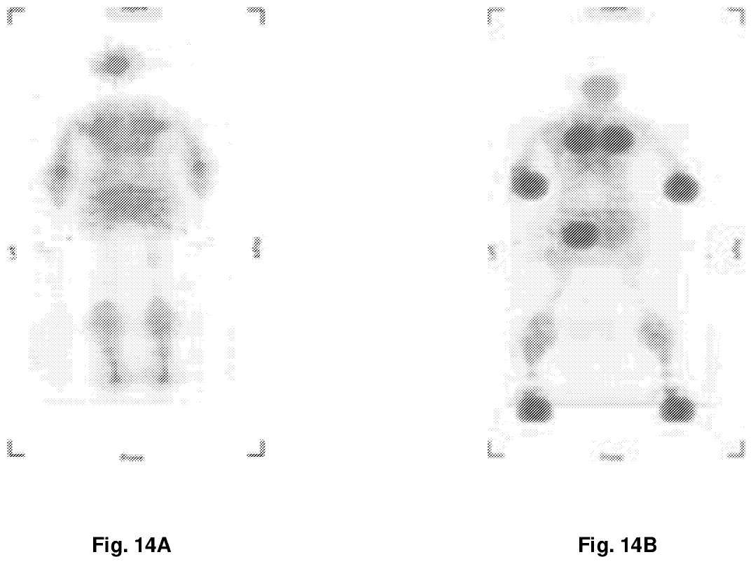

Using either method described in relation to FIG. 6 or 9 to sample each conductor array taxel 154 of the conductor array 150, the pressure sensing sheet system 100 can use the acquired absolute or relative pressure measurements to generate a heat map as shown in FIGS. 14A and 14B, for example. In some embodiments, the acquired absolute or relative pressure measurements are transmitted to server 195 for processing and generation of the heat map. The above method also allows for inherent fault checking of the conductor array 150 and the attached sheet-edge row 130 and sheet-edge column 140. Due to the output of the final shift register 310 SR_RTN 303b of both the sheet-edge row 130 and sheet-edge column 140 being used as a return signal to the MCU 510, the lack of this signal at the completion of a raster cycle would indicate a fault. The fault detection is not component specific, but for the entire conductor array 150, sheet-edge row 130, and sheet-edge column 140 assembly. If during initialisation the MCU 510 does not receive a return signal from either sheet-edge row 130 or sheet-edge column 140, it will determine that a fault has occurred once the maximum possible conductor array 150 size has been rastered. In some embodiments, the maximum possible conductor array 150 size data may be retrieved from the EEPROM 415 of sheet interface board 120 on initialisation of the pressure sensing sheet system 100.

While embodiments have been described in the context of a pressure sensing conductor array, it will be appreciated that the described embodiments can be implemented for any and all types of conductor arrays and is not limited to the applications described herein.

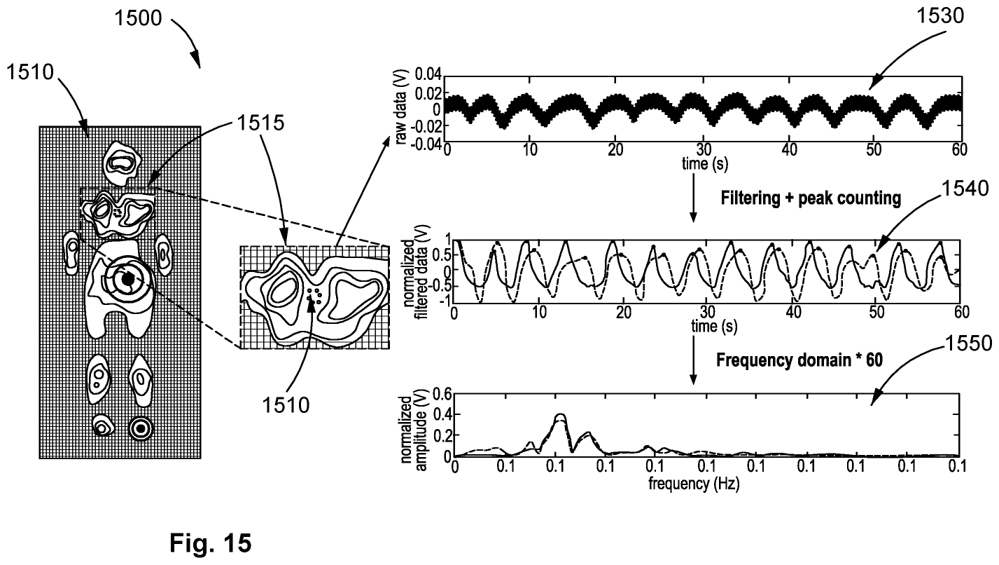

In some embodiments, the pressure sensing sheet system 100 may be used to measure patient vital signs, such as respiratory rate and heart rate, for example. FIG. 15 illustrates a method 1500 for extracting vital signs from pressure data generated pressure sensing sheet system 100. Pressure map 1510 illustrates the levels of pressure being sensed by pressure sensing sheet system 100. By using an image recognition algorithm, a chest cavity area 1515 of the pressure map 1510 can be identified. A midpoint 1520 of the chest cavity area 1515 is identified, and pressure data for midpoint 1520 is extracted for a given time period, as shown by signal 1530. Raw signal data 1530 is filtered within specific cut-off frequencies based on known physiological boundaries of vital signs. For example, the signal may be filtered within 0.16 Hz to 0.66 Hz for determining a respiratory rate which tends to be between 10 and 40 breaths per minute, or to between 0.83 Hz and 2.5 Hz for heart rate, which tends to be between 50 and 150 beats per minute. The recorded and filtered data can be analysed in both the time domain and the frequency domain. In the time domain, moving windows which may be around 1 minute in duration can be extracted and the number of peaks in that time period can be counted, to determine values for heart rate and respiratory rate, as shown by signal 1540. In the frequency domain, the frequency with the highest peak can be identified and multiplied by 60 to determine values for heart rate and respiratory rate, as shown by signal 1550.

In some embodiments, pressure sensing sheet system 100 may be used to determine whether a patient is in or out of bed. This may be done by determining whether the pressure sensing sheet system 100 senses an arbitrary weight, such as at least 10 kg. The weight sensed may be determined based on the pressure values recorded at each taxel and the number of taxels or area covered by taxels sensing a pressure. In some embodiments, pressure sensing sheet system 100 may be used to determine whether a patient has fallen out of a bed (a fall event). This may be done by using a machine learning model trained to recognise heat map patterns indicating either a fall event or a non-fall event. The model may be trained using heat map patterns associated with fall events, either simulated or real, as well as heat map patterns associated with non-fall events, for example. That is, heat map patterns captured over a period prior, during, and after both fall and non-fall events are used to train the machine learning model to recognise or predict a fall event. In some embodiments, image recognition analysis may be used in combination with the machine learning model to recognise or predict both fall and non-fall events.

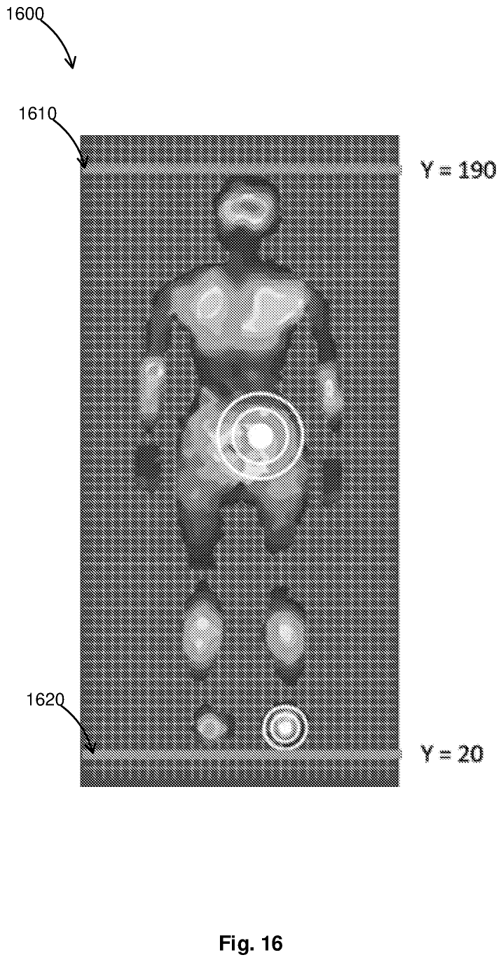

In some embodiments, pressure sensing sheet system 100 may be used to determine a patient height while the patient is lying supine on conductor array 150. This may be done by identifying the upper and lower bounds of pressure readings on the conductor array 150, subtracting the lower bound from the upper bound, and multiplying the result by the set unit spacing between each conductor array taxel 154 in conductor array 150. For example, in the pressure map 1600 shown in FIG. 16, upper bound 1610 has been measured as 190, while lower bound 1620 has been measured as 20. The difference between the upper and lower bound is 170. Assuming the spacing between each conductor array taxel 154 is 1 cm, the height of the patient can therefore be determined to be 170 cm, for example. In some embodiments, patient height may be determined using an image recognition algorithm, configured to compare a pressure heat map to a known conductor array 150 length. This alternate method of determining patient height may allow for variation in conductive fabric pitch tolerances, for example.

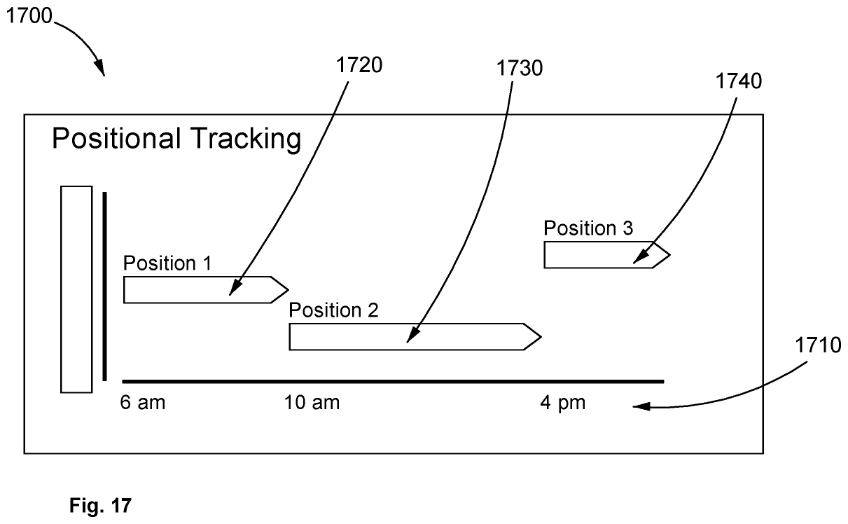

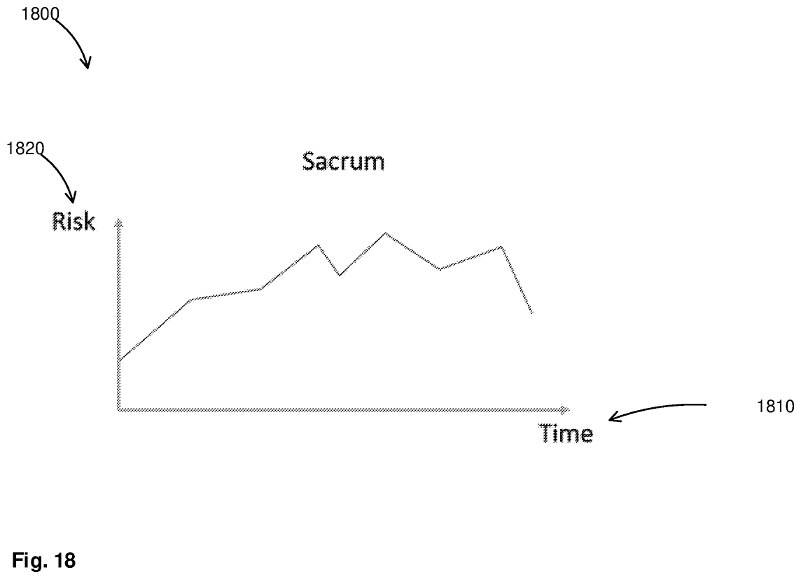

In some embodiments, the pressure sensing sheet system 100 may be used as a position or movement tracking tool. Movement tracking involves recording a history of the pressure profile as measured by conductor array 150. A snapshot of the pressure profile may be taken periodically, which can be used to determine the history of pressure over a set period of time to allow for the positions adopted by the patient or the frequency of movement of the patient to be determined. This can be used to monitor restlessness in bed, sleep activity, and other characteristics. A graphical representation of the positions adopted by a patient may be generated by using image recognition of a pressure map image generated based on the pressure measurement data. An example of such a graphical representation 1700 is shown in FIG. 17, with an x-axis 1710 showing the time, and bars 1720, 1730 and 1740 corresponding to particular positions adopted by the patient over the time period. In some embodiments, the position or movement tracking tool may be configured to recognise particular body parts of a patient in contact with conductor array 150. The position or movement tracking tool configured to recognise particular body parts may be used to perform injury risk analysis for the particular body part. That is, the probability of an injury or illness associated with the pressure applied to the specific body part may be determined, for example.

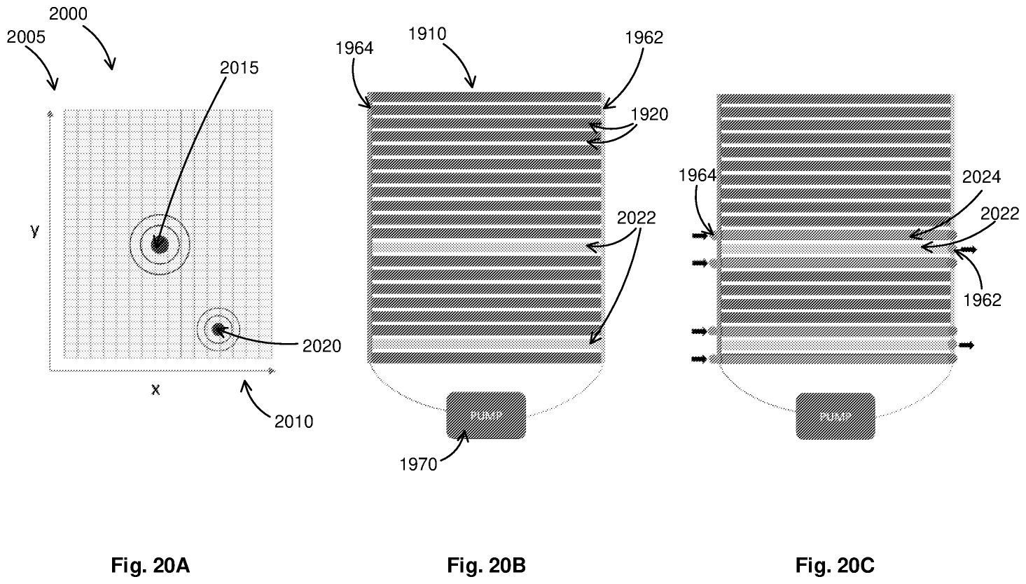

By tracking pressure data over time, levels of risk indicating how likely a patient is to develop a pressure injury in a particular area of the body may also be determined and graphically represented. For example, FIG. 20 shows a graph 1800 having an x-axis 1810 showing time and a y-axis 1820 showing a level of risk, with the risk of a pressure injury developing in the sacral region shown over time.

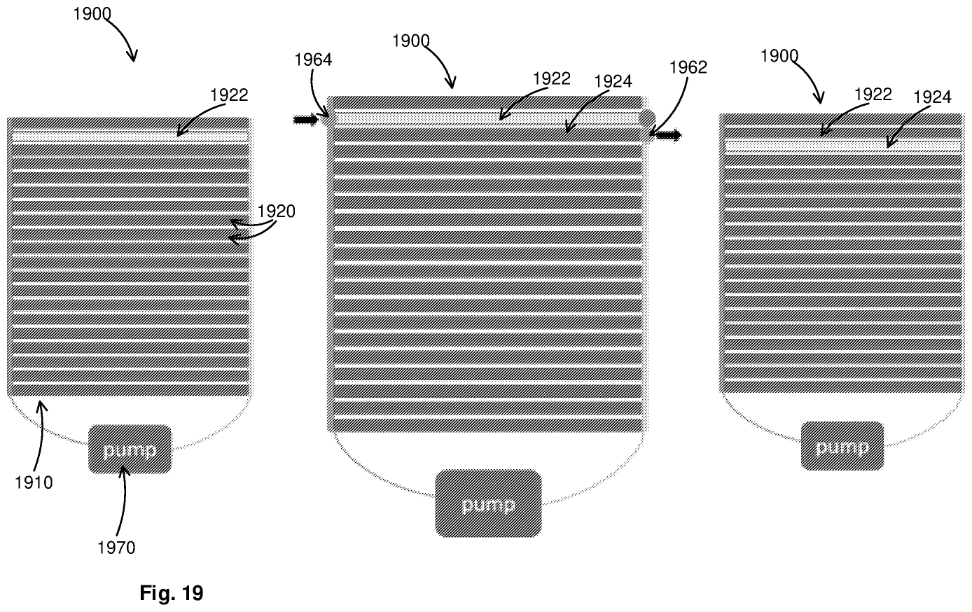

A specific application of a pressure sensing sheet system 100, according to some embodiments, used for pressure sensing is described in further detail below with reference to FIGS. 19, 20 and 21. FIGS. 19 and 20 relate to a method and system of dynamically operating an air mattress based on output produced by pressure sensing sheet system 100, to relieve pressure in areas that have been identified as at risk of causing a pressure injury to a patient occupying the air mattress. In some embodiments, the below described application may be implemented in full or in part, by an alternate control system external, or additionally, to pressure sensing sheet system 100.

FIG. 19 shows a method of controlling pressure within air-cells of an air mattress, being a method 1900 of cyclically controlling pressure in air-cells 1920 of an air mattress 1910. Air mattress 1910 comprises a plurality of air-cells 1920 spanning the length of the mattress, with the pressure within each air-cell 1920 controlled by one-way valves 1960. Valves 1960 operate on a timer, such that every timer cycle, an outlet valve 1962 opens to deflate an air-cell 1920, while an inlet valve 1964 is opened to inflate an adjacent air-cell 1920 using pump 1970. For example, at a first step of method 1900, an air-cell 1922 is deflated. At a second step, air-cell 1942 is inflated by valve 1964, while an adjacent air-cell 1924 is deflated via valve 1962. At a third step, air-cell 1922 has been inflated, while air-cell 1924 is now deflated. Some alternative methods of controlling pressure within air-cells may use pressure sensors on each air-cell, maintaining and redistributing levels of air pressure so that each air-cell is at an equal pressure.

In some embodiments, pressure sensing sheet system 100 may allow for dynamic control of air pressure within air-cells 1920 of an air mattress 1910, using conductor array 150 to sense pressure placed on areas of air mattress 1910. Pressure sensing sheet system 100 may be adaptive to the specific pressure profile being sensed, allowing for pressure to be increased and decreased throughout air-cells 1920 as required to provide targeted pressure relief In some embodiments, MCU 510 of databox 110 communicates with valves 1960 and pump 1970, causing air-cells 1920 to be inflated and deflated as desired. By integrating pressure sensing sheet system 100 with air mattress 1910, each air-cell 1920 can be correlated with a range of conductor array taxels 154 corresponding to the grid of conductor array 150. According to some embodiments, mattress 1910 may have air-cells 1920 that span one or both of the x and y axis.

An example of the above described system is shown in FIGS. 20A to 20C. FIG. 20A shows an example pressure map 2000 generated based on output received from conductor array 150 located on air mattress 1910. Pressure map 2000 has an x axis 2010 correlating to conductor columns 152 of conductor array 150, and a y axis 2005 correlating to conductor rows 151 of conductor array 150. A first zone of risk 2015 and a second zone or risk 2020 are shown as circles on pressure map 2000. Zones of risk 2015 and 2020 may have been determined based on a level of pressure sensed by pressure sensing sheet system 100 in those zones, a duration of time for which the pressure levels were sensed, pressure gradients, and other risk factors such as Braden scores, Norton scores, Waterlow scores, diabetic status, smoker status, age, BMI and other co-morbidities of the patient. Zones of risk may be identified and correlated with an x, y position of the zone within conductor array 150, such as by producing pressure map 2000. Pressure sensing sheet system 100 may then determine which air-cells 1920 correlate with the x, y positions of the zones of risk. This may be done through application programming interface (API) integration between pressure sensing sheet system 100 and air mattress 1910. Following, pressure sensing sheet system 100 can then be determined which air-cells 1920 should be inflated and/or deflated, and corresponding instruction signals may be sent to valves 1962 and 1964, and to pump 1970 to implement the instructions.

FIG. 20B shows an example air mattress 1910 corresponding to pressure map 2000. Air-cells 1922 correspond to zones of risk 1915 and 1920. Pressure sensing sheet system 100 may therefore determine that the air pressure within air-cells 1922 should be decreased, to avoid a pressure injury forming on the patient occupying air mattress 1910. FIG. 20C shows an example correction operation being performed by valves 1962 and 1964, and pump 1970, based on control signals received from pressure sensing sheet system 100. Specifically, output valves 1962 of air-cells 2022 are opened to allow air-cells 2022 to deflate, reducing the pressure on zones of risk 2015 and 2020. Input values 1964 of air-cells 2024 adjacent to cells 2022 are also opened, to allow inflation of cells 2024, to alleviate the pressure produced by cells 2022.

Further examples of applications of pressure sensing sheet system 100, according to some embodiments, may include, but is not limited to, sleep monitoring, lymphedema diagnoses, and seizure detection, for example.

In some embodiments, pressure sensing sheet system 100 may output data signals for the purpose of display by an external input/output (I/O) device capable of conveying the outputs, such as information, to a user. In some embodiments, pressure sensing sheet system 100 may include an input/output (I/O) device capable of conveying the outputs, such as information, to a user.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A pressure sensing sheet system, including:
a pressure sensing mat, including:
a piezoresistive material;
an at least one conductor array row and an at least one conductor array column, wherein the piezoresistive material is sandwiched between the at least one conductor array row and the at least one conductor array column, wherein each intersection of the conductor array row and the conductor array column defines a taxel;
at least one sheet-edge row segment coupled to the at least one conductor array row;
at least one sheet-edge column segment coupled to the at least one conductor array column;
a sheet interface board connected to the at least one sheet-edge row segment and the at least one sheet-edge column segment, and configured to act as a fan-out; and
a databox connected to the sheet interface board of the pressure sensing mat via signal wires and configured to measure each taxel of the pressure sensing mat.

2. The pressure sensing sheet system of claim 1, wherein the databox measures each taxel by iterating across all conductor array rows and conductor array columns creating a circuit across each individual taxel.

3. The pressure sensing sheet system of claim 1, wherein the taxel measurement value is serialised, allowing for an at least 10 lead connection to the sheet interface board.

4. The pressure sensing sheet system of claim 1, wherein the at least one sheet-edge row segment can drive a minimum of three and a maximum of eight conductor array rows; and
wherein the at least one sheet-edge column segment can drive a minimum of three and a maximum of eight conductor array columns.

5. (canceled)

6. The pressure sensing sheet system of claim 1, wherein both the at least one sheet-edge row segment and the at least one sheet-edge column segment are designed to chain unidirectionally with additional sheet-edge row segments or sheet-edge column segments, respectively.

7. The pressure sensing sheet system of claim 1, wherein the databox is configured to communicate with an external server to transmit measurements for processing.

8. The pressure sensing sheet system of claim 1, wherein the databox can dynamically determine the size of the conductor array.

9. The pressure sensing sheet system of claim 1, wherein the databox is configured to fault check the conductor array.

10. The pressure sensing sheet system of claim 1, wherein the databox and the sheet interface board are configured to measure multiple taxels at once to increase bandwidth.

11. The pressure sensing sheet system of claim 1, wherein the at least one sheet-edge row segment and the at least one sheet-edge column segment are configured to allow the databox to measure at least one of an absolute resistance at each taxel of the conductor array, and a relative potential difference across each taxel of the conductor array.

12. (canceled)

13. The pressure sensing sheet system of claim 1, wherein the databox is further configured to display information representative of the pressure sensing sheet system.

14. The pressure sensing sheet system of claim 1, wherein the databox is further configured to output a display signal for the purpose of displaying information representative of to the pressure sensing sheet system.

15. A method for determining pressure across a plurality of conductive rows and a plurality of conductive columns of a conductive fabric, the method comprising:
applying a first voltage supply to each row of the plurality of rows and each column of the plurality of columns of the conductive fabric;
selecting a first of the plurality of rows and a first of the plurality of columns of the conductive fabric, wherein the selected row is provided a second voltage supply greater than the first voltage supply;
measuring an output of the selected column, the output comprising an electrical signal;
iterating the selected column from the first to a last of the plurality of columns, wherein for each iteration of the plurality of columns the measuring of the output of the selected column is repeated;
iterating the selected row from the first to a last of the plurality of rows, wherein for each iteration of the plurality of rows the iteration of the columns is repeated; and
determining pressures applied to the conductive fabric based on the measured outputs.

16. The method of claim 15, further comprising using the determined pressures applied to the conductive fabric to generate a heat map or a plurality of heat maps.

17. (canceled)

18. A method for determining a risk of pressure injuries in a patient using a plurality of conductive rows and a plurality of conductive columns of a conductive fabric, the method comprising:
applying a first voltage supply to each row of the plurality of rows and each column of the plurality of columns of the conductive fabric;
selecting a first of the plurality of rows and a first of the plurality of columns of the conductive fabric, wherein the selected row is provided a second voltage supply greater than the first voltage supply;
measuring an output of the selected column, the output comprising an electrical signal;
iterating the selected column from the first to a last of the plurality of columns, wherein for each iteration of the plurality of columns the measuring of the output of the selected column is repeated;
iterating the selected row from the first to a last of the plurality of rows, wherein for each iteration of the plurality of rows the iteration of the columns is repeated;
determining pressures applied to the conductive fabric based on the measured outputs;
generating, based on the pressures applied to the conductive fabric, a plurality of heat maps; and
determining using a machine learning method, based on the plurality of heat maps, if the patient is at risk of developing a pressure injury.

19. The method of claim 15, further comprising determining a risk of a patient falling out of a bed by:
generating, based on the pressure applied to the conductive fabric, a plurality of heat maps; and
determining using a machine learning method, based on the plurality of heat maps, if the patient is at risk of falling out of the bed.

20. The method of claim 15, further including, prior to applying the first voltage supply, a method for determining the number of conductive rows in the plurality of conductive rows, the method comprising:
applying an electrical pulse to a first row shift register of a plurality of row shift registers for a single clock cycle, wherein each bit of the plurality of row shift registers is indicative of one of the plurality of conductive rows and the plurality of row shift registers are connected in series;
applying a plurality of row clock cycle signals to the plurality of row shift registers, wherein for every row clock cycle signal applied the electrical pulse shifts one bit in the plurality of row shift registers;
receiving from a last row shift register of the plurality of row shift registers the electrical pulse; and
determining the number of conductive rows in the plurality of rows based on the number of row clock cycle signals applied.

21. The method of claim 15, further including, prior to applying the first voltage supply, a method for determining the number of conductive columns in the plurality of conductive columns, the method comprising:
applying an electrical pulse to a first column shift register of a plurality of column shift registers for a single clock cycle, wherein each bit of the plurality of column shift registers is indicative of one of the plurality of conductive columns and the plurality of column shift registers are connected in series;
applying a plurality of column clock cycle signals to the plurality of column shift registers, wherein for every column clock cycle signal applied the electrical pulse shifts one bit in the plurality of column shift registers;
receiving from a last column shift register of the plurality of column shift registers the electrical pulse; and
determining the number of conductive columns in the plurality of columns based on the number of column clock cycle signals applied.

22. The method of claim 15, further including determining the number of conductive rows and the number of conductive columns based on data stored in a memory.

23. The method of claim 20, further including a method for fault checking by determining if a fault is present based on the electrical pulse, wherein not receiving the electrical pulse after the determined number of clock cycle signals has been applied is indicative of a fault.

24. (canceled)

25. (canceled)

26. (canceled)
